(19) United States
(12) Patent Application Publication
(10) Pub. No.: US 2017/0111490 A1
(43) Pub. Date: Apr. 20, 2017

(54) METHOD OF TRANSMITTING DATA AND A DEVICE AND SYSTEM THEREOF

(71) Applicant: ActivWell Limited, Shatin (HK)

(72) Inventors: Chor Tin Ma, Shatin (HK); Ming Yip Wallace Wong, Shatin (HK)

(73) Assignee: ActivWell Limited

(21) Appl. No.: 15/394,183

(22) Filed: Dec. 29, 2016

Related U.S. Application Data
(63) Continuation-in-part of application No. 14/596,644, filed on Jan. 14, 2015.

(51) Int. Cl. H04M 1/725; A61B 5/00; A61B 5/024; H04B 1/69; G08C 17/02

(52) U.S. Cl. CPC H04M 1/7253; H04B 1/69; G08C 17/02; A61B 5/024; A61B 5/0008

(57) ABSTRACT

A method for transmitting data signal from a sending-end 100 to a receiving-end 200, comprising the steps of spreading the data signal into a spread spectrum signal, combining the spread signal with an analogue signal into a mixed signal. The mixed signal can be transmitted or broadcasted to the receiving-end 200. The receiving-end is able to play or use the analogue signal without significant noise from the spread spectrum signal. The spread spectrum signal can be retrieved by inverse-spread-processing the spread spectrum signal to retrieve the data signal. The sending-end can be in a headphone 900 and the receiving-end can be in a mobile phone 910, or the sending-end can be a loudspeaker broadcasting the analogue signal to a mobile phone picking up the analogue signal by the microphone in the mobile phone.

The present invention relates to communication technology, and particularly relates to a method and system for analogue signal transmission, and more particularly between a mobile phone and a headphone or a speaker system and receiving terminal.

### BACKGROUND

Mobile phones and similar mobile phone devices have an audio jack into which the connector of a headphone may be plugged for the wearer to listen to music played in the mobile phone. The headphone typically comprises a microphone, and a set of earphones for being placed on the ears of the wearer. The wearer can engage in a conversation wearing the headphone, by listening to a conversation party by the ear phones and by speaking into the microphone.

The standard audio jack on mobile phones is a 3.5 mm audio jack. This audio jack is dedicated to wired transmission of analogue audio signals between the mobile phone and the headphone, particularly from the microphone to the mobile phone and from the mobile phone to the ear phones. The audio jack is unuseable for transmission of non-analogue audio signal such as data, since the audio jack is dedicated to analogue audio signal transmission for tele-conversation.

It is desirable to provide a way of expanding the use of the wired 3.5 mm audio jack to increase functions of a conventional mobile phone.

Furthermore, it is desirable to provide a way by which any analogue audio signal transmitter can be used to transmit non-conversation or non-audio data.

### SUMMARY OF THE INVENTION

In a first aspect, the invention proposes a method of transmitting data between a mobile phone and a headphone plugged to the analogue audio jack of the mobile phone, comprising the steps of: providing a data signal; spreading the data signal to produce a spread spectrum signal having a wider bandwidth than the data signal and a smaller power spectral density than the data signal; sending the spread spectrum signal from the mobile phone to the headphone and the inverse-spread-processing the spread spectrum signal in the headphone to retrieve the data signal; or sending the spread spectrum signal from the headphone to the mobile phone and the inverse-spread-processing the spread spectrum signal in the mobile phone to retrieve the data signal.

Optionally, there is no need for the spread spectrum signal to have a wider bandwidth than the data signal and a smaller power spectral density than the data signal, although this is preferable.

Preferably, the method further comprises the steps of: providing an analogue signal; combining the analogue signal and spread spectrum signal into a mixed signal; wherein the spread spectrum signal is sent from the mobile phone to the headphone as part of the mixed signal; or the spread spectrum signal is sent from the headphone to the mobile phone as part of the mixed signal.

Advantageously, the invention provides the possibility that a conventional audio jack in mobile phones can be used to transmit to a headphone, or earphone, an analogue audio signal and a data signal at the same time via the same channel of the audio jack. This expands the use of the conventional audio jack which has until now been dedicated to only analogue audio signal transmission. Now, all that is needed in some embodiments is a headphone which can collect a desired data signal, and which can spread the data signal to produce a spread spectrum signal, and combine the spread spectrum signal with an analogue audio signal recorded by the microphone. The data signal can be any information, typically biological data such as heart rate or temperature of the wearer, which is obtained by sensors suitably installed as part of the headphone. The combination produces a mix signal of the spread spectrum signal and the analogue audio signal. Subsequently, a complementary mobile phone which has been enabled to despread the mix signal receives the mixed signal through the audio jack and despreads the mixed signal. The despreading process restores and extracts the data signal from the mixed signal. As the skilled man knows, the same despreading process is able to remove or reduce the presence of the analogue audio signal in the retrieved data signal, so that the data signal suffers only insignificant amount of noise or none at all.

The reverse configuration is also possible, where a conventional audio jack in a mobile phone can be used to transmit a mixed signal to the headphone. In this configuration, an analogue audio signal and a piece of data signal are mixed in the same way: the data signal is spread and mixed with the analogue audio signal, and sent by the audio jack to the headphone. This allows a headphone to be controlled by signals sent via the audio jack, which is not possible in prior art. The analogue audio signal can be a piece of music played in the mobile phone or radio broadcasts received in radio frequency from the ambient surroundings.

Typically, the data signal contains information to be recorded, processed, played or displayed in the mobile phone.

In a variation of the embodiment, it is possible that two or more data signals are provided at the same time in the same mixed signal, along with the analogue audio signal, and both of which can be extracted by the mobile phone. A copy of the mixed signal can be despread using one despread code, and a copy of the same mixed signal can be despread using another despread code, so that the two data signals can be distinguished from each other.

The skilled man understands that by ‘data signal’, the term is only used to identify a signal which carries specific information or data, and data signal can also itself be any suitable analogue signal, even an analogue audio signal or a digital signal.

By ‘analogue signal’, this term refers to a signal into which a spread spectrum signal is mixed, and the spread spectrum signal being generally unnoticeable by the target person receiving information based on the analogue signal until the spread spectrum signal is despread and retrieved from the mixed signal. Optional terminology may refer to the ‘analogue signal’ as a ‘carrier signal’ and the ‘spread spectrum signal’ as a ‘hidden’ or ‘carried signal’.

Preferably, the method further comprises the steps of: the data signal triggering operation of a function in the headphone when the mixed signal is sent from the mobile phone to the headphone and the inverse-spread-processing the spread spectrum signal in the headphone to retrieve the data signal; or the data signal triggering operation of a function in the mobile phone when the mixed signal is sent from the headphone to the mobile phone and the inverse-spread-processing the spread spectrum signal in the mobile phone to retrieve the data signal.

Therefore, a mobile phone can now control any function in a headphone by sending a control or instruction signal via the same audio jack and headphone wire, such as to trigger the headphone to monitor heartbeat or temperature of the wearer. This again expands the functions of the audio jack in conventional mobile phones.

Preferably, the audio jack is a 3.5 mm audio jack. This specifically expands the use-ability of the conventional 3.5 mm audio jack.

In a second aspect, the invention proposes a method for transmitting data signal from an analogue signal sending-end to an analogue signal receiving-end, comprising the steps of: providing a data signal; spread processing the data signal to produce a spread spectrum signal having a wider bandwidth than the data signal and a smaller power spectral density than the data signal; sending the spread spectrum signal to the receiving end; inverse-spread-processing the spread spectrum signal to retrieve the data signal.

The sending-end and the receiving-end can be defined as such in any two respective device which communicates an analogue signal between them, typically in wired connection but possibly wirelessly as well. The sending-end can be the headphone as mentioned and the receiving-end a mobile phone or vice versa. The sending-end can be a loudspeaker broadcasting an audio signal to the ambient surroundings, a light source, a microwave source and so on. In different applications, such analogue signals can be used to hide useful message or alerts. For example, a microwave oven cooking food using microwave can have a data signal is hidden in the microwave in the form of a spread spectrum signal. A terminal device which can detect microwave can be used to despread the data signal and issue a warning of microwave leaking into the surroundings.

In a third aspect, the invention proposes a method for transmitting data signal from an analogue signal sending-end to an analogue signal receiving-end, comprising the further steps of: providing an analogue signal; combining the analogue signal and the spread spectrum signal into a mixed signal; wherein sending the spread spectrum signal to the receiving end includes sending the mixed signal to the receiving end.

In a fourth aspect, the invention proposes a device for transmission of analogue audio signal comprising a sensor for obtaining a data signal; the device capable of code spreading the data signal to produce a spread spectrum signal, the spread spectrum signal having a frequency bandwidth broader than the bandwidth of the data signal and having an power spectral density lesser than the power spectral density of the data signal; wherein the device is capable of transmitting the spread spectrum signal.

Typically, the device is capable of obtaining an analogue audio signal; capable of adding the spread spectrum signal to the analogue audio signal to provide a mixed signal; and capable of transmitting the spread spectrum signal as part of the mixed signal.

Transmission of an analogue signal refers taking an analogue signal and sending it from a transmission end or sending end, to a receiving end, and includes different modes of transmission.

For example, the analogue signal can be digitized and transmitted in such a digitized form to the receiving end, and be converted back into the analogue signal at the receiving end. The same applies to the mixed signal comprising the analogue signal and the data signal.

Preferably, the device is capable of transmitting the spread spectrum signal by an analogue signal jack.

Optionally, the device is a microphone. Alternatively, the device is an earphone or headphone. Alternatively, the device is an earphone or headphone having an integral microphone.

Typically, the sensor is a heart rate sensor, a temperature sensor, a gyrometer or an accelerometer, or any combination thereof.

In a fifth aspect, the invention proposes a new use of a mobile phone having an audio jack, wherein the audio jack is suitable for receiving a spread data signal which has been code spread, and the mobile phone for despreading the spread data signal and obtaining the despread data signal as an instruction for operating a function of the mobile phone.

In a sixth aspect, the invention proposes a loudspeaker system for sending data signal, such as text messages wherein the loudspeaker system is capable of playing an audible analogue signal mixed with a spread spectrum signal; the spread spectrum signal being a data signal which have been spread processed to have a relatively low spectral power compared to the audible analogue signal, and the data signal representing the text message; and the spread spectrum signal is suitable for despreading to retrieve the data signal by a device capable of receiving the audible analogue signal mixed with a spread spectrum signal.

Therefore, the invention provides the possibility that ambient analogue signals broadcasted into the general surroundings of a premises, such as analogue audio signals from a loudspeaker system, can be used to carry data signals to all devices capable of picking up these ambient analogue signals. It is possible in some applications that the analogue signal is not audio but a source of light, or other radio frequency signals or even microwave signals that can be broadcasted to receiving devices and despread to retrieved data signals mixed into the analogue signal.

In a seventh aspect, the invention proposes a device suitable for receiving a spread spectrum signal; wherein the device is capable of detecting the spread spectrum signal when the device apply a despreading process on the spread spectrum signal.

Preferably, the device receives the spread spectrum signal as part of a mixed signal, the mixed signal comprising the spread spectrum signal and an analogue signal; wherein the device has a receiver capable of receiving analogue signals; and the spread spectrum signal is detected when the device apply a despreading process on the analogue signal.

Typically, wherein the analogue signal is an analogue audio signal, although it may be other analogue signals such as radio frequency, microwave or even visible radio frequency.

Typically, the spread spectrum signal contains information on biological or physiological data of a human or living thing.

Typically, the device is a headphone or a mobile terminal. ‘Mobile terminal’ here refers to mobile phones, personal digital assistants, computer notebooks, any form of computer tablets or smart phones, and so on, suitable for receiving the mixed signals.

In another aspect, the invention proposes a data signal transmission method comprising the steps of: a transmitting side to obtain the data signal and the analog analogue audio signal; wherein the transmission side of the data signal by the spread spectrum processing to obtain a spread spectrum signal; the end of the transmitted spread spectrum signal the analog analogue audio signals into the mixed signal and sent to the receiver; the receiver receiving the mixed signal, the data signal to obtain said mixed signal by inverse spreading processing.

Preferably, said step of transmitting said data terminal through spread spectrum processing the signal to obtain a spread spectrum signal is: the transmitting terminal via the preset spreading code of the data signal to obtain a direct sequence spread spectrum signal spread spectrum processing.

Preferably, said spreading code is an orthogonal spreading code; preset by the transmitting side of the data signal spreading code direct sequence spread Step frequency spread spectrum signal is processed to obtain: the transmitting side through the preset orthogonal spreading codes with the data signal is multiplied to obtain the spread spectrum signal.

Preferably, said receiving step by the end of the mixing signal to obtain the inverse spread spectrum processing of the data signal is: the receiving end of the positive the spreading code and the post down-mix signal is multiplied; the receiving terminal by multiplying the signal obtained by said obtaining said data signal.

The data signal transmission method as described characterized in that said step of said transmitting side to the receiving side of the mixed signal is transmitted to: the transmitting side through the audio jack of the mixed signal is transmitted to the receiving end.

Preferably, the audio jack is a standard wireline audio jack; the transmitting side through the step of mixing the audio jack signal is transmitted to the receiving end is: the transmission end of the cable through the audio jack standard of the mixed signal is transmitted to the receiver.

Preferably, said standard audio jack is a wired 3.5 mm audio jack; the sending end through a standard interface to the wireline analogue audio signals to the step of mixing the receiving end is: the transmitting side through the audio jack 3.5_mixed signal is transmitted to the receiving end.

Preferably, said sending side, through the step of mixing the data signal and analogue audio signal, is transmitted to the receiving end by modulating the mixed analogue signal with radio frequency

Preferably, the bandwidth of the data signal less than the threshold value. This is to ensure the spread spectrum signal's power spectral density is low enough to be not noticeable.

In a further aspect, the invention proposes a data signal transmission system, including sending and receiving ends, characterized in that said transmitting side comprising: a signal-acquisition-module for acquiring the data signal and the analog analogue audio signal; spread-spectrum-processing-module is used by the processing said data signal is spread to obtain spread spectrum signals; signal synthesis module for converting the spread spectrum signal and analogue audio signals into the mixed signal; means for transmitting the mixed signal is transmitted to the receiving end; said receiving end comprises: a signal-receiving-module for receiving the mixed signal; the reverse spreading processing module for obtaining the data signal by the mixed signal inverse spreading processing.

Preferably, the transmission system, characterized in that said processing module is further configured to spread by a preset spreading code direct sequence spread spectrum processing to obtain the spread spectrum signal to the data signal.

Preferably, said spreading code is an orthogonal spreading code; wherein the processing module is further spread by an orthogonal spreading code and a preset. data signal is multiplied to obtain the spread spectrum signal.

Preferably, said processing module is further configured to reverse spreading the orthogonal spreading code and the mixed signal is multiplied by the multiplying the resulting signal to obtain the data signal.

Preferably, said signal transmitting module is further used by the audio jack of the mixed signal is transmitted to the receiver.

Preferably, the audio jack is an audio jack standard; the signal transmitting module is further used by the audio jack standard wired transmitting the mixed signal to the receiver.

Preferably, the audio jack standard wired 3.5 mm audio jack; module is further configured to send the signal through a 3.5 mm audio jack of the mixed signal is transmitted to the receiving end.

Preferably, said signal transmitting module is further used after the mixed signal transmitted through the wireless transmission modulation scheme to the receiver side.

Preferably, the bandwidth of the data signal is less than the threshold value.

In a further aspect, the invention proposes a signal transmission control method comprising: a mobile phone terminal is able to terminate the data transmission; said mobile phone terminal generate a feedback signal to the peripheral device. When the peripheral device receives the feedback signal, stop sending data signals.

Preferably, said method further comprising: said mobile phone terminal is able to recover the signal transmission; said mobile phone terminal terminates a feedback signal is generated; in the detection of the peripheral devices. When the feedback signal is not received or the received feedback signal and the mixed signal does not correspond, restore the transmission data signal.

Preferably, said method further comprising: said peripheral device receives the data transmission to recover the signal; resume transmission of said peripheral device data signals.

Preferably, said method further comprising: said peripheral device through the audio input interface of the mixed signal is transmitted to the mobile phone terminal.

Preferably, said mobile phone terminal transmits the feedback signal to the step of the peripheral device is as follows: the mobile phone terminal through the audio output interface to return the feedback signal to the peripheral device.

Preferably, said receiving terminal terminates the data transmission signal prior to the above step further comprising: said mobile phone terminal detects whether there is an incoming call; the mobile phone terminal detects an incoming call and generating the data transfer termination signal; the mobile phone terminal returns a feedback by: the mobile phone terminal acquires the incoming call voice analog analogue audio signal and transmits the feedback signal to after the synthesis setting means; step of said peripheral device receives said feedback signal is: the peripheral device by the received signal to obtain said feedback signal.

Preferably, said mobile phone terminal is able to, prior to the above step, resume the signal transmission, further comprising: if the mobile phone terminal detects an incoming call hangs up; the mobile phone terminal generates a recovery signal.

In another aspect, the invention proposes a signal transmission system control system including a mobile phone terminal and a peripheral device, wherein said mobile phone terminal comprising: an instruction acquisition module for acquiring data transfer termination signal; a feedback signal generation module for receiving mixed signal to generate said feedback signal corresponding to the mixed signal, the mixed signal comprising the identification signal and a data signal and/or analog analogue audio signal; a feedback module for the feedback signal back to said mobile phone terminal; said peripheral device comprising: a signal transmission module for transmitting the mixed signal to said mobile phone terminal; signal-to-signal ratio of the feedback module for mixing the received signal with the comparison, the feedback When a signal waveform corresponding to the mixed signal to notify termination of the signal transmitting module transmits the data signal.

Preferably, the instructions to obtain the data acquisition module is further configured to restore the transmission signal, and notifies the termination of the feedback signal generation module generating a feedback signal; a ratio of the signal module is further operable to detect when the feedback signal is not received or the received signal and the feedback signal does not correspond to the mixing, the notification signal transmitting module to resume transmission of the data signal.

Preferably, said peripheral apparatus further includes an instruction-receiving module, configured to receive a data transmission resume signal, the notification signal transmitting module to resume transmission of the data signal.

Preferably, said signal transmitting module is further used by the audio input interface of the mixed signal is transmitted to the mobile phone terminal.

Preferably, the signal via the audio output module is further configured to interface to the feedback signal to the peripheral device.

Preferably, said mobile phone terminal further comprises call detection module for detecting whether there is an incoming call, generating a data transfer termination signal upon detection of an incoming call; said mobile phone terminal further comprising a signal synthesis module, for acquiring voice calls analog analogue audio signal and the feedback signal synthesizer; said peripheral device further comprises a signal filter module for passing the received signal to obtain said feedback signal.

Preferably, said detection module is further configured to detect the call is an incoming call hangs up, the data generated in the transmission resuming signal is detected when an incoming call hangs up.

In a further aspect, the invention proposes a method of transmitting data from a headphone to a remote mobile phone, wherein the headphone comprises at least one sensor for obtaining physiological data of a person wearing the headphone, comprising the steps of: the at least one sensor obtaining a physiological data signal; spreading the physiological data signal to produce a spread spectrum signal; the headphone having a microphone, the microphone obtaining an analogue signal; combining the analogue audio signal and the spread spectrum signal into a mixed signal; wherein the spread spectrum signal is sent from the headphone to a proximate mobile phone, and the proximate mobile phone in turn sending the mixed signal to the remote mobile phone by telecommunication channels.

This provides the advantage of remote reception of physiological data of the person wearing the headphone. Remote medical consultation, including consultation on health matters, dietary matters is made possible.

Preferably, the headphone is plugged to an analogue audio jack of the remote mobile phone; and the audio jack is a wired 3.5 mm audio jack, which is typically found in virtually all older models of mobile phone.

Most areas requiring remote medical consultation tend to be in third world regions where the latest smart phones may not be available. However, older models of mobile phones have pervasively reached many remote geographical regions. Therefore, by using an existing mobile phone technology, older mobile phones can be given a new lease of life, reducing the need for state of the art resources to provide medical consultation to people in remote areas of the world. The use of existing mobile jacks for data communication at the same time as one is having a conversation via the same mobile jack points the way towards a direction which is not contemplated by in the prior art.

Preferably, the at least one sensor obtaining a physiological data signal is a heart rate detector suitable for obtaining the heart rate of the person wearing the headphone.

Preferably, the at least one sensor obtaining a physiological data signal is a temperature monitor suitable for obtaining the temperature of the person wearing the headphone.

In a further aspect, the invention proposes a method of receiving data in a computing device, comprising the steps of: the computing device receiving telecommunication signal; the telecommunication signal being a mixed signal of an audio signal and a spread spectrum signal; the computing device inverse-spread-processing the spread spectrum signal to retrieve a physiological data signal.

This provides the advantage that a computer or a mobile phone is able to receive physiological data of a person by receiving an audio signal carrying the physiological data. A person is able to talk to another person in a remote location at the same time as receiving the physiological data.

Preferably, the physiological data signal represents the heart rate of a person. Preferably, the physiological data signal represents the temperature of the person.

Typically, the computing device is a mobile phone, and the telecommunication signal is transmitted from a remote mobile phone; the remote mobile phone being in communication with a headphone; the headphone having at least one sensor for obtaining the physiological data from the person wearing the headphone; the telecommunication signal provided according to the steps of: the at least one sensor obtaining the physiological data signal; spreading the physiological data signal to produce a spread spectrum signal; the headphone having a microphone, the microphone obtaining an analogue signal; combining the analogue audio signal and the spread spectrum signal into a mixed signal; wherein the spread spectrum signal is sent from the headphone to the remote mobile phone, and the remote mobile phone in turn sending the mixed signal to the computing device as the telecommunication signal.

### BRIEF DESCRIPTION OF THE DRAWINGS

FIG. 1 is a flowchart of a data signal transmission method in an embodiment;

FIG. 2 is a schematic diagram of spectrum changes before and after spread spectrum processing of a data signal in an embodiment;

FIG. 3 is a structure diagram of physical audio jack plugs in an embodiment;

FIG. 4 is a structure diagram of a data signal transmission system in an embodiment;

FIG. 4A is an illustration of a headphone according to an embodiment of the invention;

FIG. 5 is a structure diagram of a data signal transmission system in another embodiment;

FIG. 6 is a flowchart of a signal transmission control method in an embodiment;

FIG. 7 is an illustration of yet another embodiment; and

FIG. 8 illustrates yet another embodiment of the invention.

### DETAILED DESCRIPTION

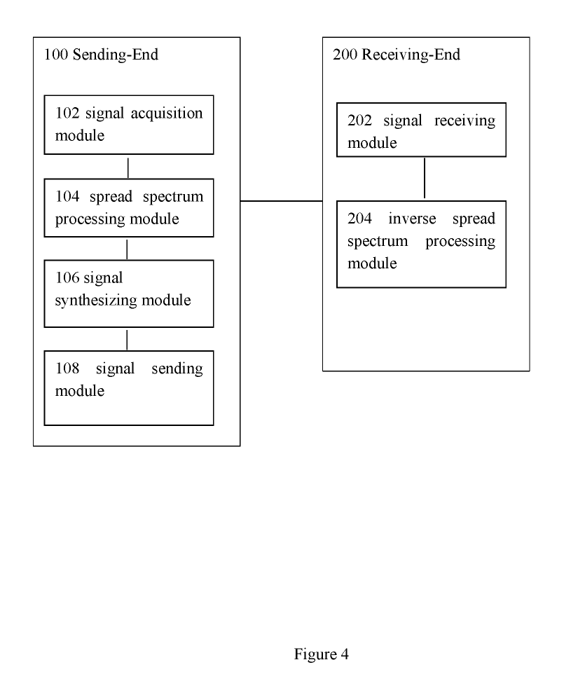

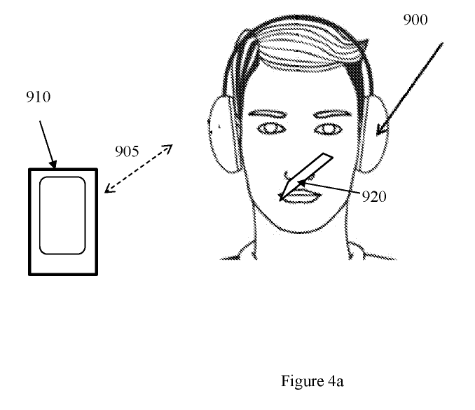

FIG. 4A shows a multi-functional headphone 900 which is capable of communicating, at 905, with a mobile phone 910.

The headphone 900 has ear phones which are placed over the ears of the wearer, so that the wearer may listen to analogue audio signal transmission from the mobile phone 910.

The headphone 900 also has a microphone 920 into which the wearer may speak, so that his speech can be converted into an analogue audio signal and transmitted to the mobile phone 910, to be further transmitted by a telecommunication operator to a conversation party. An analogue audio signal typically has a frequency spectrum ranging between 20 Hz to 20 kHz.

Provided in the ear phones is at least one sensor (not visible in FIG. 4A). In this first embodiment, the sensor is a heartbeat sensor capable of monitoring the pulsation of blood flow in the ear. The heartbeat sensor is therefore able to collect data in the form data signal of the pulsation indicating the wearer's heart rate. Typically, the data signal is a pulse signal where each pulse represents one heartbeat. Each pulse signal has a narrower frequency band than analogue audio signals captured by the microphone 920 from the voice of the wearer. The headphone 900 sends the pulse signals to the mobile phone 910 for heart rate monitoring, such as for use by exercise monitoring applications in the mobile phone. Typically, each pulse signal has a bandwidth below a pre-determined threshold, which is preferably, 4 kHz. In addition, the data signal has smaller power spectral density and also a smaller data size than that of the analogue audio signal. The data size is typically less than 10 bytes/second.

Optionally, however, the sensor may be replaced by anyone of or any combination of two or more sensors, such as the heartbeat sensor, a body temperature sensor, a blood pressure sensor or a motion sensors etc. The motion sensor can be a single axis or multi-axis gyrometer or an accelerometer. Therefore, data signals collected by the headphone 900 are typically biological or physiological data such as heart rate, body temperature, blood pressure, movements etc.

Typically, a wire extends from the headphone 900 ends in a phone connector suitable for being plugged into a 3.5 mm audio jack or audio jack. That is, a physical wire connects the multi-function headphone 900 to a mobile phone 910.

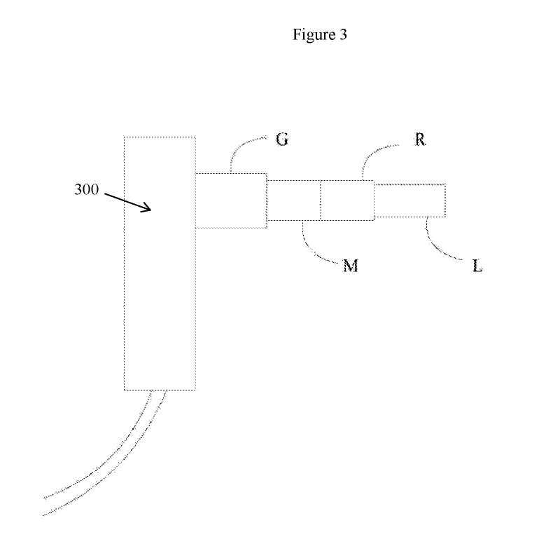

FIG. 3 shows a standard 3.5 mm audio jack phone connector 300 comprising four analogue channels, L, R, G and M. L and R are the respective left and right channels of the audio jack, M is the microphone 920 channel, and G is ground.

Typically, analogue signals are sent from the mobile phone 910 to the headphone 900 by channels L and R, so that the wearer may listen to the analogue audio signals. Channels L and R are therefore usually uni-directional from the mobile phone 910 to the headphone 900.

Analogue signals are sent from the microphone 920 to the headphone 900 by channel M, so that the wearer's speech may be transmitted to the mobile phone 910 to be sent to the conversation party. Channel M is therefore usually uni-directional from the microphone 920 to the mobile phone 910.

FIG. 4 is a schematic diagram of a data signal transmission system between the multi-function headphone 900 and the mobile phone 910. To transmit an analogue audio signal from the microphone 920 to the mobile phone 910, there is a sending-end 100 defined in the multi-function headphone 900 and a receiving-end 200 in the mobile phone 910. The sending-end 100 and the receiving-end 200 communicate by the wire connecting the headphone 900 to the mobile phone 910.

The sending-end 100 comprises a signal-acquisition-module 102, a spread-spectrum-processing-module 104, a signal-synthesizing-module 106 and a signal-sending-module 108.

The receiving-end 200, on the other hand, comprises a signal-receiving-module 202 for receiving the mixed signal and an inverse spread-spectrum-processing-module 204. The inverse spread-spectrum-processing-module 204 is for carrying out inverse spread spectrum processing on the mixed signal to provide a data signal.

In a best mode embodiment, the signal-acquisition-module 102 is collecting data signal representing the wearer's heart rate at the same time as the wearer of the headphone 900 is speaking into the microphone 920. Therefore, both an analogue audio signal of the speech and a data signal of the heart rate have to be transmitted to the mobile phone 910.

The spread-spectrum-processing-module 104 therefore multiplies the data signal with a pre-determined spreading code to produce a ‘spread spectrum signal’. A spreading code is typically a high-rate spread spectrum sequence. Typically, the bandwidth of the data signal is relatively narrow before being multiplied or spread with the spreading code. Multiplying the spreading code with the data signal converts the data signal from a narrow bandwidth signal into a broad bandwidth signal, that is, a signal which is very much more spread out in the frequency domain that the original data signal, and possibly shifted to be over a range of higher frequencies. The multiplication is typically done via a multiplication circuit (not illustrated). Preferably, the spread-spectrum-processing-module 104 carries out Direct Sequence Spread Spectrum (DSSS) processing on the data signal. Optionally, however, the spread-spectrum-processing-module 104 carries out the spread spectrum processing by Orthogonal Code Spread Spectrum (OCSS), Frequency-Hopping Spread Spectrum (FHSS) and so on.

The spread-spectrum-processing-module 104 performs the spreading by phase-shift keying (PSK) of the data signal. In this case, the carrier frequency of the PSK is preferably 18 khz. The highest frequency on the spectrum of the spread spectrum signal is typically 22.1 khz.

However, the analogue audio signal is not treated by the spreading code.

Subsequently, the signal-synthesizing-module 106 synthesizes the spread spectrum signal and the analogue audio signal into a ‘mixed signal’, such as by adding the spread spectrum signal to the analogue audio signal using an addition circuit (not illustrated).

The signal-sending-module 108 then transmits this mixed signal to the receiving-end 200.

In the prior art, a mobile phone 910 is unable to handle more than one signal at the audio jack. In this embodiment, however, both the spread spectrum signal and the analogue audio signal are transferred as one combined analogue signal from the headphone 900 to the mobile phone 910, through the same wire.

Therefore, the signal-receiving-module 202 in the receiving-end 200 receives the mixed signal. The inverse spread-spectrum-processing-module 204 then captures an accurate phase of the spreading code through a synchronization capture module and thereby generates a spreading code phase which is identical with the phase of the spreading code at the sending-end 100. This generated spreading code phase is a local despread spectrum signal′. The local despread spectrum signal can be multiplied with the mixed signal to retrieve the data signal.

The despreading is a mathematical treatment which will suppress the analogue signal while extracting data signal, and there is no need to separate the spread spectrum signal from the analogue audio signal in the mixed signal in order to extract the data signal.

In the event that the signal-acquisition-module 102 fails to acquire an analogue audio signal, such as when the wearer is not speaking into the microphone 920, the signal-sending-module 108 will send only the spread spectrum signal to the receiving-end 200. The receiving-end 200 will simply continue to receive information on the wearer's heart rate.

The heart rate may be used by the mobile phone 910 to monitor the state of health or exertion of the wearer. On the other hand, if the signal-acquisition-module 102 fails to acquire a data signal, such as if the sensor monitoring heart rate malfunctions, the signal-sending-module 108 will send only the analogue audio signal to the receiving-end 200 so that any tele-conversation can be continued without disruption.

Optionally, the mixed signal is encoded or modulated before transmission from the sending-end. In this case, the inverse spread-spectrum-processing-module 204 also comprises capability for decoding and/or demodulating the mixed signal before despreading.

Therefore, the embodiment as described allows two sources of information to be sent as one mixed signal though an audio jack between a headphone (or any device) to a mobile phone (or any complementary device).

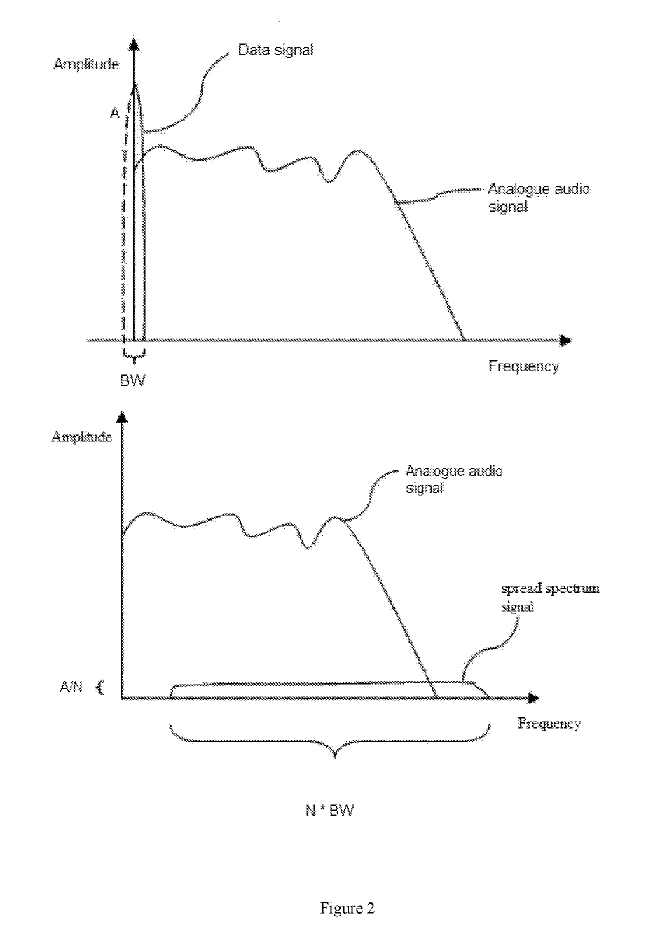

FIG. 2 shows two power spectral density-frequency charts, illustrating the power distribution of the signal which is transmitted from the sending-end 100. The part of the top chart indicated ‘A’ is the power spectral density of the data signal. The lower chart has a portion marked ‘N’, which is the spread spectrum gain. ‘BW’ indicates the frequency bandwidth. The charts illustrate an audible signal within the typical range of generally 20 hz˜20 khz.

The top chart shows a data signal having a narrow bandwidth about the x-axis representing frequency and also a high power. The same chart shows an analogue audio signal which is spread over a large bandwidth and having high power, as is typical for human voice.

The bottom chart shows the data signal after it has been spread processed using the spreading code, converting the data signal into a spread spectrum signal, having now a broad bandwidth with a lower spectra power.

On one hand, the analogue audio signal between 20 hz˜20 khz may be regarded as strong noise in the spread spectrum signal. Such strong noise at 20 hz˜20 khz causes relatively low interference in the spread spectrum signal on the whole when the spread spectrum signal is despread. This allows the despread data signal retrieved from the mixed signal to have low bit error rate and to suffer less impact from the analogue audio signal; despreading of the mixed signal will diminish the presence of the analogue audio signal while retrieving the data signal.

On the other hand, as the spread spectrum signal has a wider frequency range and that it has a lower power spectral density within the range of 20 hz˜20 khz, the spread spectrum signal may be regarded as very low level noise in the analogue audio signal. In particular, the signal-synthesizing-module 106 has preferably adjusted the power spectral density of the spread spectrum signal before generating the mixed signal. Therefore, the spread spectrum signal is inaudible even if the receiving-end 200 receives and plays the mixed signal directly through an audio play device, without filtering away the spread spectrum signal. For example, the mobile phone may play aloud the speech of the wearer which he speaks into the microphone, and the data signals indicating the wearer's heart rate is undetectable by the a listener. This is because the audible range to the human ear is about 20 hz˜20 khz, and the spread spectrum signal has a wide spectrum range that may stretch over 20 khz but has a low power spectral density within the range of 20 hz˜20 khz.

More specifically, the low interference effect from the spread spectrum signal on the analogue audio signal is provided because the signal-synthesizing-module 106 adjusts the power of the spread spectrum signal, such that the spread spectrum signal is below a pre-determined ratio to the power of the analogue audio signal. Alternatively, the signal-synthesizing-module 106 simply adjusts the power of the spread spectrum signal until it is below an absolute power threshold level.

Furthermore, even if the mixed signal comprising the analogue audio signal is encoded into a digital analogue audio signal or directly input to an audio play device, the contribution of the spread data signal within the range of 20 hz˜20 khz is relatively low.

Prior to this embodiment, conventional use of the audio jack is limited in use to only analogue audio signal transmission from the headphone 900 to the mobile phone 910. The audio jack was not used to transfer any information other than analogue audio signals through the same wire. If the data signal were not spread processed, sending the data signal through the same audio wire as the analogue audio signal will create an annoying noise which can be heard by conversation party at the other side of the telephone call.

Therefore, the preset embodiment provides an expanded and extended use of the audio jack. The resulting advantage is that many older version mobile phones 910 having only this audio jack and no other ports for data communication is now able to process more information than just an analogue audio signal. Owners of older mobile phones 910 may now find their mobile phone capabilities expanded to more uses. All that needs to be upgraded is the software in these older phones to perform despreading and providing a headphone 900 with spread processing functions.

In a variation of the embodiment, the sending-end and the receiving-end are reversed in location; the sending-end 100 is set in the mobile phone 910 instead of the headphone 900 and the receiving-end 200 is set in the headphone 900 instead of the mobile phone 910. In this case, the data signal is a control instruction generated in the mobile phone 910. The control instruction is processed by multiplying it with the spreading code, and then mixed with an analogue audio signal incoming into the mobile phone 910. The mixed signal is sent to the headphone 900 so that the wearer can listen to the analogue audio signal. The control instruction may be a button-triggered instruction for controlling a sensor switch regulating the volume in the multi-function headphone 900. As in the earlier embodiment, the data signal corresponding to the control instruction is preferably a pulse signal with a bandwidth below the threshold. Optionally, instead of an incoming analogue audio signal which is part of a tele-conversation, the analogue audio signal is of a piece of music played on the mobile phone 910 transmitted the headphone 900.

Therefore, an embodiment has been described in which two sets of information are sent through a common audio signal wire at the same time, but without causing interference to the analogue audio signal which the wearer hears or which is transmitted to the conversation party.

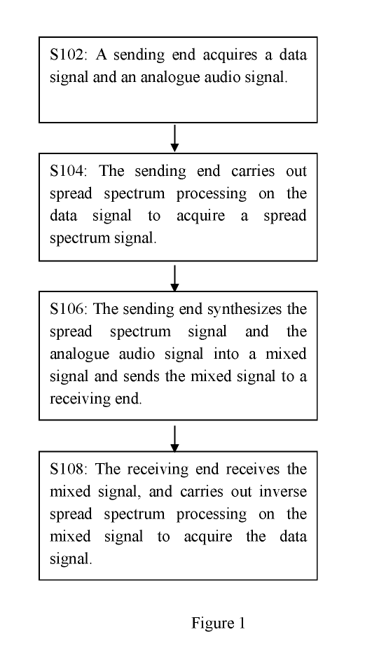

FIG. 1 is a flowchart showing the steps executed by the described embodiment in obtaining and transmitting the data signal.

At Step S102, the sending-end 100 acquires a data signal and an analogue audio signal. The sending-end 100 in this case refers to the headphone 900, which comprises the microphone 920 and the sensor in the headphone providing signals to be transmitted to the mobile phone 910. Therefore, an analogue audio signal such as a voice signal is collected by the microphone 920 while a data signal is collected by the sensor representing the wearer's heart rate.

At Step S104, spread spectrum processing is performed on the data signal at the sending-end 100 to produce a spread spectrum signal.

At Step S106, the spread spectrum signal and the analogue audio signal are synthesized, or combined, into a mixed signal, and transmitted from the sending-end 100 to the receiving-end 200.

At Step S108, the receiving-end 200, i.e. the mobile phone 910, receives the mixed signal and carries out inverse spread spectrum processing on the mixed signal in order to retrieve the data signal. Specifically, this comprises a step of the receiving-end 200 firstly capturing an accurate phase of the sent spreading code through a synchronization capture module of the spreading code. The receiving-end then generates a spreading code phase which is identical with the spreading code phase at the sending-end 100, and which is use-able as a local despread spectrum signal. Subsequently, the receiving-end 200 multiplies the local despread spectrum signal by the mixed signal. This restores the spread spectrum signal to its original data signal, in the original bandwidth and power state. There is no need to separate the spread spectrum signal and the analogue audio signal before applying the despread spectrum signal. Optionally, the local despread spectrum signal is pre-determined and pre-stored in the mobile phone 910 for despreading spread spectrum signal from the headphone 900.

Preferably, the spreading code used at the sending-end 100 is an orthogonal spreading code. In this case, the inverse spread spectrum processing simply comprises multiplying the orthogonal spreading code and the mixed signal at the receiving-end 200. Optionally, the spread spectrum signal is filtered to retrieve the data signal more accurately.

As mentioned, the signal-synthesizing-module 106 adjusts the power of the spread spectrum signal to a pre-determined ratio to the power of the analogue audio signal. This ensures any contribution of noise by the spread spectrum signal to the analogue audio signal is kept below a certain threshold. Alternatively, the signal-synthesizing-module 106 adjusts the overall power of the spread spectrum signal to be below a certain threshold regardless of the power of the analogue audio signal, which also limits noise contribution from the spread spectrum signal. In this way, the sending-end 100 ensures noise power in the analogue audio signal contributed by the spread spectrum signal is kept to a limit, making it possible for the receiving-end 200 to play the mixed signal directly to produce a virtually or practically noise-free audible sound to a listener, without having to remove or filter away the spread spectrum signal first.

In the above embodiments, the sending-end 100 is defined as the headphone 900, where the user's heart rate is collected as well as his speech, both to be transmitted to the mobile phone 910. In this case, the headphone 900 sends the mixed signal to the receiving-end 200 through the M channel. However, when the mobile phone 910 sends an analogue audio signal from the mobile phone 910 to the headphone 900, the sending-end 100 is defined as being located on the mobile phone 910 and the receiving-end 200 on the multi-function headphone 900. In this case, the sending-end 100 sends an analogue audio signal, or a mixed signal synthesized from the analogue audio signal and the spread spectrum signal, to the receiving-end 200 through the L and/or R channel.

In other possible embodiments, the sending-end 100 sends the mixed signal to the receiving-end 200 by means of wireless transmission after modulating the mixed signal. The form of wireless transmission may be Bluetooth, infrared, etc. The mixed signal may be firstly modulated with a relatively high carrier frequency (2.4 ghz in the case of Bluetooth), transmitted and then demodulated, and acquired.

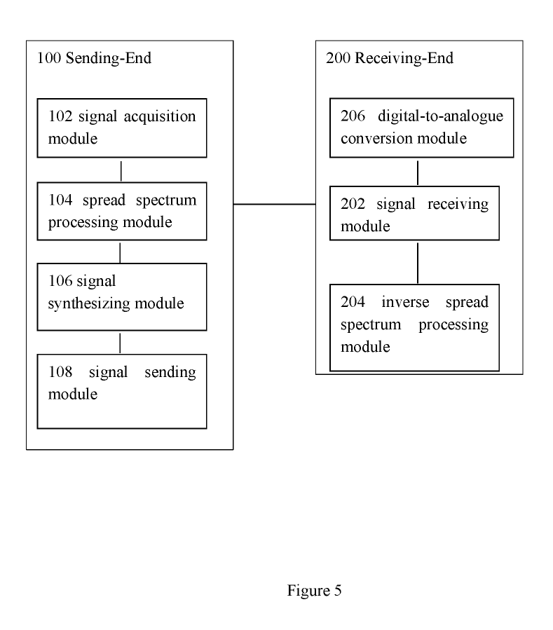

FIG. 5 is a schematic diagram of a variation of the embodiment of FIG. 4, wherein the receiving-end 200 further comprises a digital-to-analogue conversion module 206. The digital-to-analogue conversion module 206 is capable of encoding the received mixed signal into a digital analogue audio signal with PCM (Pulse-code modulation) encoding. A digital analogue audio signal obtained by this encoding is used as digital input to mobile phone 910 software applications.

In most conventional mobile phone 910 and headphone 900 designs, the headphone 900 has less computing power than the mobile phone 910. That is, the headphone 900 having processor which has a lower computing capacity then the processor. Therefore, most conventional mobile phone 910 and headphone 900 have unequal processing power. In this case, it will be easier for a data signal to be spread processed in the mobile phone 910 than to be spread processed in the headphone 900, as spreading requires more processing than despreading. Therefore, to avoid over burdening the processor in the headphone 900, the headphone 900 only collects data signal when there is no on-going mobile phone 910 conversation. If a mobile phone 910 call comes in to the mobile phone 910, and an analogue audio signal from a conversation party has to be transmitted to the headphone 900, the headphone 900 will stop collecting data signal and concentrate on processing the analogue audio signal to let the wearer listen to the conversation party and to reply by the microphone 920. Such a method of conserving processing power in the headphone 900 requires the headphone 900 to be alerted whenever a mobile phone 910 call is coming in, and alerted whenever the call is hung up.

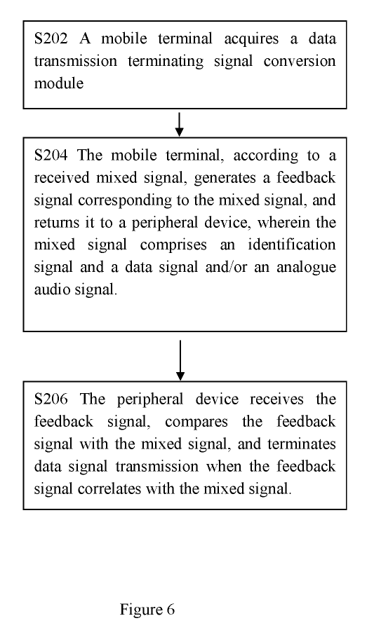

FIG. 6 shows how to alert the headphone 900 to stop collecting data signal when a telephone call comes in, which comprises the following steps at the receiving-end 200.

In Step S202, the mobile phone 910 detects an incoming call.

Subsequently, in Step S204, the mobile phone 910 generates a feedback signal for sending from the mobile phone 910 to the headphone 900. The feedback signal is generated by using the same signal which is being transmitted from the headphone 900 to the mobile phone 910. That same signal is processed to have a reduced power spectral density. The feedback signal is therefore like an echo of the analogue audio signal being transmitted to the mobile phone 910.

Subsequently, in Step S206, the multifunctional headphone 900 receives the feedback signal. The multifunctional headphone 900 compares the feedback signal with the mixed being sent signal, and the two signals would be almost identical. Therefore, the feedback signal correlates with the mixed signal. On detecting the correlation, the multifunctional headphone 900 terminates data signal transmission, or stops collecting data signals, or stops combining the data signal with the analogue audio signal. Therefore, this embodiment uses the wire to send information from the mobile phone 910 to the headphone 900 for controlling headphone 900 functions, without needing other input or output ports; only the conventional audio signal jack is used. When the mobile phone 910 stops sending the feedback signal, the multifunctional headphone 900 resumes data signal transmission, and continues to collect data signal and combine the data signal with the analogue audio signal.

In a variation of the embodiment, instead of using the feedback signal to indicate to the headphone 900 to stop data signal transmission, an identification signal is sent to the headphone 900 continually to let the headphone 900 know to continue acquiring data signal, spreading the data signal and sending the data signal to the mobile phone 910 through the analogue audio wire. Detection of this identification signal means that there is no mobile phone 910 conversation and the headphone 900 continues to collect data signal. If the mobile phone 910 detects an incoming call, this identification signal is no longer sent by the mobile phone 910 to the headphone 900. The headphone 900 detects absence of the identification signal and stops collecting or transmitting data signal.

Optionally, the mobile phone 910 is also capable of detecting whether the incoming call has hung up and, if so, generates a data-transmission-restoring-signal. In this way, the mobile phone 910 is able to inform the multifunctional headphone 900 in real time to restore data signal transmission.

In the event the headphone 900 is transmitting heart rate signals to the mobile phone 910, but the wearer is not engaging in a conversation, but the mobile phone 910 detects an incoming telephone call, the mobile phone 910 generates a data-transmission-terminating-signal upon detecting the incoming call.

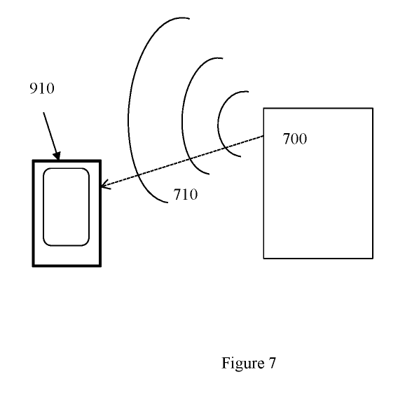

Other embodiments of sending data signals via an analogue signal channel for controlling devices are possible. For example, as shown in FIG. 7, the microphone (not the aforementioned microphone 920 in the headphone 900) of a mobile phone 910 into which a person speaks can be set to pick up an analogue signal broadcasted into the surroundings by a loudspeaker system 700. This ‘ambient’ analogue signal broadcasted, at 710, into the surroundings can be a piece of music, or news reading and so on. The loudspeaker system 700 can be one of those which blast announcement or music in a large hall. Typically, a data signal which has been spread and transformed into a spread spectrum signal is mixed with the analogue audio signal, and the mixed signal is broadcasted. The human ear cannot detect the spread spectrum signal. The data signal can embody a text message. The mobile phone 910 is configured to perform a despreading on all received analogue audio signal to see if a data signal mixed into the analogue audio signal can be retrieved. If the data signal is retrieved, the text message is displayed in the screen of mobile phone 910. In this way, a text message can be broadcasted by a loud speaker as part of an analogue audio signal, detected by all mobile phones 910 within detection vicinity and displayed by the mobile phones 910. Accordingly, this embodiment allows analogue signals containing a text message to be broadcasted together with analogue audio signals, such that the text message can be retrieved by any device capable of despreading the mixed signal. This embodiment will find application in helping people who are hard of hearing to be kept updated by the text messages with important audio announcements being made through a public announcement system.

Optionally, the text message can be replaced by an instruction which instructs the device to do something or perform a function.

The embodiments as discussed above only represent several modes of implementing the present invention. Despite the specific and detailed descriptions, these embodiments are not to be understood as limiting the scope of the present invention thereto on such basis. It should be pointed out that persons of ordinary skills in the art, without departing from the conception of the present invention, would further make a number of transformations and improvements within the scope of protection of the present invention. Therefore, the scope of protection of the present invention shall be determined by the attached claims.

For example, although a mobile phone which has an analogue audio jack has been mentioned in the description of embodiments, any device which can receive an analogue signal can be used in place of the mobile phone. This includes any device which can receive analogue audio signals such as a computer notebook, a personal digital assistant (PDA), any form of smart phones as the case may be.

Also the analogue signal can be transmitted wirelessly without need of a wire. Also, the analogue signal can be any analogue signal that can be picked up by the device, even in the radiofrequency domain, microwave domain, visible light domain, invisible light domain and so on. Also, it is possible in some embodiments that the analogue signal is converted to digital signals for transmission from the sending end to the receiving end, and to be restored from the digital to analogue at the receiving end before despreading is applied. In some other embodiments, the digitized signal can be directly despread without the signal having been re-converted into analogue, as this only depends only the mathematical treatment which the skilled man can design.

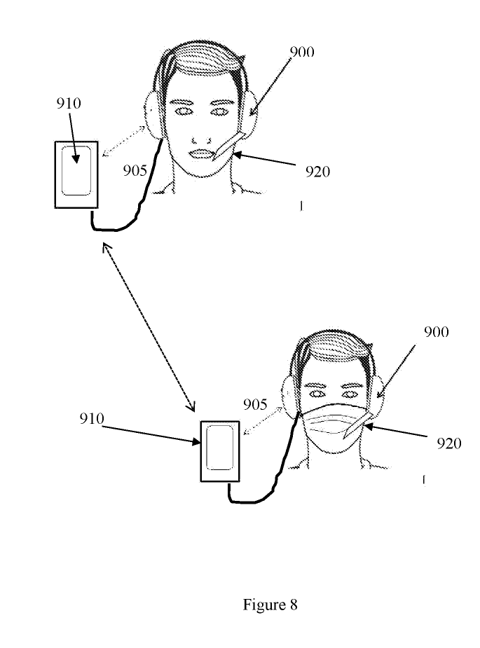

FIG. 8 illustrate a further embodiment, showing a user of the embodiment wearing an earpiece which communicates with his mobile phone. The connection of the earpieces to the mobile phones is a wire connection to the mobile phones 3.5 mm jack. Physiological data is obtained by a sensor in an ear piece or headphone, as described in the earlier embodiments. The physiological data signal is spread and mixed with audio signal from the earpiece's microphone and sent to the mobile phone. When the mobile phone receive the mixed signal sent by the earpiece, the mobile phone further sends the mixed signal through to the conversation other party by known telecommunication channels. The mobile phone of the other party already expects that there may be data signal mixed into the audio signal. and has the necessary configuration to de-spread the mixed signal to retrieve the data signal. In this way, the user and the other party are able to engage in a conversation while yet transmitting data from the user to the other party through the mobile phone 3.5 mm jack. Typically, the other conversation party is a doctor, a nurse or some other medical personnel. Different sensors installed into the earpiece allow the medical personnel to make observations or diagnosis based on the data and recommend treatment immediately. Such an earpiece may be used with people who needs to be monitored regularly but time and distance do not offer them access to medical supervision normally, such as elderly people living in remote or mountainous regions, astronauts, nomadic people and so on.

Where the other party is using a mobile phone to converse with the user, the mobile phone may be installed with an application or software which can interpret or display the de-spread data signal in real-time to the other party. Optionally, the other party receives the mixed signal via a device such as a computer instead of a mobile phone, which will allow the other party to engage in a conversation with the user nevertheless but a computer hooked up to a database is able to record the data signal into the database or immediately retrieve historical data by which to compare and review the data signal in real-time.

This embodiment therefore expands the use of conventional mobile phones, allowing them to be upgraded into remote, real-time medical consultation devices without need to modify the hardware of the mobile phones. The embodiment provides an advantage over any other type of remote analysis or diagnosis as the user is able to engage in live consultation with a service provider regarding his physiological data. Furthermore, the embodiment provides a discreet manner of obtaining diagnostic data of the user, as an ear piece is not very conspicuous in modern society. This allows the user to obtain live diagnosis as required, such as blood oxygen level when he is asleep, his heart rate when running, and all these may be monitored live by medical personnel through the earpiece.

In variations of the embodiment, the earpiece is not connected to a 3.5 mm jack in a mobile phone. However, in order to optimise the benefits made possible by the embodiment, an existing audio jack which heretofore has been used only for audio analogue signal transmission may now be used to transmit more than analogue data, lending a new lease of life to the device. In particular, many rural and third world countries use older models of second hand phones. It is particularly in these rural areas that medical service are lacking or wanting, mainly due to inaccessibility. To be able to provide medical consultation through an ear piece using existing technology, without needing the latest connection ports or the latest smartphone might throw a lifeline to some of the people living in these areas.

1. A method of transmitting data from a headphone to a remote mobile phone, wherein the headphone comprises at least one sensor for obtaining physiological data of a person wearing the headphone, comprising the steps of:
the at least one sensor obtaining a physiological data signal;
spreading the physiological data signal to produce a spread spectrum signal;
the headphone having a microphone, the microphone obtaining an analogue signal;
combining the analogue audio signal and the spread spectrum signal into a mixed signal;
wherein
the spread spectrum signal is sent from the headphone to a proximate mobile phone, and the proximate mobile phone in turn sending the mixed signal to the remote mobile phone by telecommunication channels.

2. A method of transmitting data from a headphone to a remote mobile phone as claimed in claim 1, wherein
the headphone is plugged to an analogue audio jack of the remote mobile phone; and
the audio jack is a wired 3.5 mm audio jack.

3. A method of transmitting data from a headphone to a remote mobile phone as claimed in claim 1, wherein
the at least one sensor obtaining a physiological data signal is a heart rate detector suitable for obtaining the heart rate of the person wearing the headphone.

4. A method of transmitting data from a headphone to a remote mobile phone as claimed in claim 1, wherein
the at least one sensor obtaining a physiological data signal is a temperature monitor suitable for obtaining the temperature of the person wearing the headphone.

5. A method of receiving data in a computing device, comprising the steps of
the computing device receiving telecommunication signal;
the telecommunication signal being a mixed signal of an audio signal and a spread spectrum signal;
the computing device inverse-spread-processing the spread spectrum signal to retrieve a physiological data signal.

6. A method of receiving data in a computing device as claimed in claim 5, wherein
the physiological data signal represents the heart rate of a person.

7. A method of receiving data in a computing device as claimed in claim 5, wherein
the physiological data signal represents the temperature of the person.

8. A method of receiving data in a computing device as claimed in claim 5, wherein the computing device is a mobile phone.

9. A method of receiving data in a computing device as claimed in claim 5, wherein the telecommunication signal is transmitted from a remote mobile phone;
the remote mobile phone being in communication with a headphone;
the headphone having at least one sensor for obtaining the physiological data from the person wearing the headphone;
the telecommunication signal provided according to the steps of:
the at least one sensor obtaining the physiological data signal;
spreading the physiological data signal to produce a spread spectrum signal;
the headphone having a microphone, the microphone obtaining an analogue signal;
combining the analogue audio signal and the spread spectrum signal into a mixed signal;
wherein
the spread spectrum signal is sent from the headphone to the remote mobile phone, and the remote mobile phone in turn sending the mixed signal to the computing device as the telecommunication signal.
